(19) United States
(12) Patent Application Publication
(10) Pub. No.: US 2008/0213192 A1
(43) Pub. Date: Sep. 4, 2008

(54) AEROSOL LOTION FORMULATIONS

(75) Inventors: Marcia S. Schlesinger, Germantown, TN (US); David Scott Manley, Cordova, TN (US); Heidi N. Graham, Cordova, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc.

(21) Appl. No.: 11/769,482

(22) Filed: Jun. 27, 2007

Related U.S. Application Data
(60) Provisional application No. 60/816,955, filed on Jun. 27, 2006.

(51) Int. Cl. A61K 9/12; A61Q 17/04; A61Q 99/00

(52) U.S. Cl. 424/47

(57) ABSTRACT

Non-foaming aqueous emulsion compositions; methylparaben-based preservative systems for aqueous aerosol compositions having a pH at or above a pH where methylparaben begins to hydrolyze; cosmetic, household, and human and veterinary pharmaceutical products containing these compositions.

This application claims priority to U.S. Application Ser. No. 60/816,955 filed Jun. 27, 2006 the contents of which are hereby incorporated by reference in their entirety.

### FIELD OF THE INVENTION

The subject invention is directed to aerosol lotion formulations, in particular emulsion formulations that are held under pressure. The subject invention is also directed to aerosol formulations containing parabens as a preservative, and more particularly to the use of methylparaben as preservative in an aerosol formulation maintained at a pH above a level where the preservative normally would hydrolyze.

### BACKGROUND OF THE INVENTION

Antimicrobial preservatives are substances or preparations which destroy, or prevent or inhibit the proliferation of, microorganisms in composition, and which may also offer protection from oxidation. Preservatives are frequently used to make self-sterilizing, aqueous based cosmetic products such as emulsions. Preservatives prevent the development of microorganisms that may be in the product from growing during manufacturing and distribution of the product and during use by consumers, who may further inadvertently contaminate the products during normal use. Typical preservatives include the lower alkyl esters of para-hydroxybenzoates (parabens), especially methylparaben, propylparaben, isobutylparaben and mixtures thereof.

Parabens are a commonly used and effective preservative for aqueous formulations and extensively used in foods, drugs and cosmetics because of their lack of toxicity and broad spectrum of preservative activity. Combinations of parabens are typically used, and also used in combinations with other preservatives to provide for protection over a range of pH. For example, methylparaben is stable between pH 3 and 6, but hydrolyzes above pH 6, thereby reducing its effectiveness and usefulness in neutral to basic formulations. (Raval, N. N and Parrott, E. L., J. Pharm. Sci., Vol. 56, No. 2, 274(1967) As a result, formulators will include additional preservative ingredients in such formulations knowing that the methylparaben may not be stable, leading to over-preserved products.

Thus, it would be useful to provide aqueous formulations having a pH over 6 that are preserved with inexpensive yet effective parabens such as methylparaben instead of more expensive preservatives. Moreover, it would be useful to be able to avoid the use of additional preservatives that are typically included in aqueous formulations having a pH over 6. Further it would be useful to prepare these formulations as aerosols, particularly aerosols containing dimethyl ether, to be used in a wide array of consumer and personal care products such as hair sprays, sunscreens, insecticides, herbicides, cosmetics, and household products such as cleansers, coatings, and paints, among other uses.

Some formulations, such as traditional sunscreen formulations, can be delivered as aerosols, typically as liquid formulations held under pressure by a propellant which releases the formulation from the container when an orifice in the container is opened. However, for formulations formed as emulsions, whether as oil-in-water or oil-in-water emulsions, the presence of additional components such as surfactants and film forming agents, when combined with a propellant, tend to form a foam as it is expressed from the container due to the expansion of the gaseous propellant. Although the presence of the foam does not diminish the usefulness of the formulations, in certain product categories, foaming formulations are not a desired quality.

Thus, it would also be useful to provide aerosol formulations of emulsions that do not form foams when expressed from a container. These and other objectives are obtained by the invention more fully described and claimed herein.

### SUMMARY OF THE INVENTION

The subject invention provides an aerosol composition comprising methylparaben as a preservative and having a pH at or above a level where the methylparaben would begin to hydrolyze, wherein the methylparaben does not significantly degrade.

The invention also provides an aqueous composition under pressure comprising a propellant in admixture and methylparaben, wherein the composition maintains a pH of greater than 6 and the methylparaben does not significantly degrade.

The invention also provides a sunscreen composition comprising methylparaben in an amount sufficient to act as an antimicrobial agent, wherein the composition comprises a pH above 6 and the methylparaben does not significantly degrade, and wherein the composition is contained in a pressurized container.

The invention further provides a method for preserving an aqueous composition under pressure and having a pH over 6, comprising adding to the composition of preservative system comprising methylparaben in an antimicrobial effective amount.

The invention also provides a preservative system for an aqueous aerosol composition having a pH at or above a level where the methylparaben would begin to hydrolyze, the preservative system comprising methylparaben present in an antimicrobial effective amount.

The invention also provides a sunscreen composition comprising methylparaben in an amount sufficient to act as an antimicrobial agent, wherein the composition comprises a pH above 6 and the methylparaben does not significantly degrade and wherein the composition is contained in a container pressurized with dimethyl ether.

The invention further provides an aerosol emulsion formulated to be held in a container under pressure, wherein the emulsion formulation does not form a foam upon expression from the container.

### DETAILED DESCRIPTION

The subject invention provides an aerosol compositions, in particular aerosol emulsion compositions. As will be described more fully herein, one aspect of the invention is that the compositions comprise methylparaben as a functioning preservative and yet are formulated a pH at or above a level where the methylparaben would begin to hydrolyze. Further, another aspect of the invention is that the aerosol compositions comprise emulsions that do not form foams when expelled from a container under pressure, as would be expected from such compositions.

In the practice of the subject invention, the propellant used in the composition may be any suitable gas, or combination of gasses, that can be compressed or liquefied within a dispensing spray canister, which expand or volatilize to vapor or gas form upon exposure to ambient temperature and pressure conditions to deliver the composition in an aerosol form, and which will solubilize methylparaben. Suitable propellants include hydrocarbons having 1 to 5 carbon atoms, including but not limited to methane, ethane, propane, isopropane, butane, isobutane, butene, pentane, isopentane, neopentane, pentene, hydrofluorocarbons (HFCs), chlorofluorocarbons (CFCs), nitrogen, ethers including dimethyl ether, and any mixtures thereof. Those of ordinary skill in the art recognize that in a closed container such as an aluminum can or glass bottle, propellants such as dimethyl ether condense to the liquid state at ambient temperature. Thus, the composition in the aerosol container is liquid formulation which can contain dissolved propellant, undissolved liquid propellant and gaseous propellant. All of this is under pressure due to the vapor pressure of the propellant. In the practice of the subject invention, the propellant can be present in an amount up to about 90 weight percent, preferably from about 2 weight percent to about 50 weight percent, and more preferably about 5 weight percent to about 40 weight percent, more preferably at about 30 weight percent, based on the total weight of the aerosol composition.

Thus, the invention further provides an aqueous composition under pressure comprising a propellant in admixture and methylparaben, wherein the composition maintains a pH of greater than 6 and the methylparaben does not significantly degrade. The invention further provides a method for preserving an aqueous composition under pressure and having a pH over 6, comprising adding to the composition of preservative system comprising methylparaben in an antimicrobial effective amount. In a preferred embodiment, the methylparaben is added to the composition before it is pressurized with a propellant. In a preferred embodiment, the composition maintains a pH of greater than 7, and in an equally preferred embodiment, the composition maintains a pH of greater than 8. In the practice of the invention, the composition should remain stable such that the methylparaben does not significantly degrade for a period of at least two years.

The compositions of the invention can also comprise aerosol foams or so-called mousse compositions. For example, U.S. Pat. No. 6,627,585 describes a mousse-forming cleansing shampoo composition comprising a foamable concentrate comprising at least one surfactant, dispersed particles of a water-insoluble conditioning agent, an aqueous carrier; and an aerosol propellant. U.S. Pat. No. 6,264,964 describes a cosmetic composition including a crosslinked non-emulsifying polysiloxane elastomer and a carboxyvinyl polymer which is in the form of a aerosol foam in a pressurized system. The propellant may be introduced into the mousse composition at the time of filling by using a standard aerosol dispenser, e.g. a spray can arrangement.

The subject invention also provides a preservative system for an aqueous aerosol composition having a pH at or above a level where methylparaben would begin to hydrolyze, the preservative system comprising methylparaben present in an antimicrobial effective amount. In a preferred embodiment, the preservative system consists essentially of methyl paraben present in an antimicrobial effective amount. In an equally preferred embodiment, the preservative system consists of methyl paraben present in an antimicrobial effective amount. The preservative system has wide applicability for cosmetic, household and pharmaceuticals for human or veterinary use. Products that are known to contain parabens, in particular methylparaben, as a preservative include moisturizers, cleansers, conditioners, shampoo, body wash, styling gel/lotion, eye cream and eye liner, blush, mascara, foundation, nail polish, polish remover, eye shadow, lipstick, lip gloss, lip liners, lip balms, makeup remover, nail treatment, foot care compositions, acne treatment, redness/rosacea treatment, varicose/spider vein treatment, anti-aging compositions, sunscreens, sunless tanning compositions, after-sun compositions, concealers, hair color and bleaching compositions, skin fading/lighteners, body firming lotion, shaving cream, after shave, relaxer, antiperspirants and deodorants, exfoliants, scrubs, liquid hand soap, bubble bath, pain and wound treatment compositions, insect repellant, anti-itch and rash cream, styling mousse and foams, bath oils and salts, toothpaste, perfume, glitter, lubricants, body powder, body oil, body spray, baby lotion, diaper cream, baby soap, baby shampoo, baby oil, baby wipes, hair-loss treatment, hair spray, cuticle treatment, dandruff/scalp treatment, depilatory, hair growth inhibitors, hair removal waxes, personal cleansing, cologne, oil controller, hand sanitizer, mouthwash, tooth whitening, eye drops, and artificial tears compositions. Any aerosol formulations of these types of applications are contemplated to be within the scope of this invention.

The compositions of the present invention may contain a wide range of additional, optional components which are referred to herein as “cosmetic components”, but which can also include components generally known as pharmaceutically active agents, The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers. SPF boosters, waterproofing agents, and viscosity increasing agents (aqueous and nonaqueous).

The subject invention also provides a sunscreen composition comprising methylparaben in an amount sufficient to act as an antimicrobial agent, wherein the composition comprises a pH above 6 and the methylparaben does not significantly degrade, and wherein the composition is contained in a pressurized container. In the practice of the invention, the sunscreen composition may contain one or more sunscreen active agents. For purposes of the present invention, a “sunscreen active agent” or “sunscreen active” shall include all of those materials, singly or in combination, that are regarded as acceptable for use as active sunscreening ingredients based on their ability to absorb UV radiation. Such compounds are generally described as being UV-A, UV-B, or UV-A/UV-B active agents. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the US but are allowed in formulations sold outside of the US include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. However, as the list of approved sunscreens is currently expanding, those of ordinary skill will recognize that the invention is not limited to sunscreen active agents currently approved for human use but is readily applicable to those that may be allowed in the future.

In a preferred embodiment, the subject invention provides a sunscreen composition comprising methylparaben in an amount sufficient to act as an antimicrobial agent, wherein the composition comprises a pH above 6 and the methylparaben does not significantly degrade and wherein the composition is contained in a container pressurized with dimethyl ether. In the practice of this aspect of the invention any of the above noted sunscreen active agents are useful.

As used herein, the term “sunless-tanning” or “self-tanning compositions” refer to compositions which, when applied to human skin, impart thereto an appearance similar to that achieved by exposing the skin to natural or artificial sunlight. Examples of sunless tanning active agents are described in U.S. Pat. Nos. 6,482,397, 6,261,541, and 6,231,837. Such sunless tanning compositions typically comprise, in addition to an artificial tanning effective amount of a self tanning agent, effective amounts of a composition coloring agent and a cosmetically acceptable carrier adapted for topical application to human skin. The self tanning agents can also include those compositions generally accepted in the art for application to human skin, and which, when so applied, react therein with amino acids so as to form pigmented products Such reactions give the skin a brown appearance similar to the color obtained upon exposing it to sunlight for periods of time sufficient to tan the skin. Suitable self tanning agents include, without limitation, alpha-hydroxy aldehydes and ketones, glyceraldehyde and related alcohol aldehydes, various indoles, imidazoles and derivatives thereof, and various approved pigmentation agents Presently preferred herein as self tanning agents are the alpha-hydroxy aldehydes and ketones. Most preferably, the self tanning agent is dihydroxyacetone (“DHA”). Other suitable self tanning agents include, without limitation, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde.

The compositions of the invention can further comprise skin protectant active agents. Suitable examples include (with preferred weight percent ranges), Allantoin (0.5 to 2 percent); Aluminum hydroxide gel (0.15 to 5 percent), Calamine (1 to 25 percent); Cocoa butter (greater than 50); Cod liver oil (5 to 14 percent); Colloidal oatmeal; Dimethicone (1 to 30 percent); Glycerin (20 to 45 percent); Hard fat (greater than 50); Kaolin (4 to 20 percent); Lanolin (12.5 to 50 percent); Mineral oil (greater than 50 percent); Petrolatum (greater than 30 percent); Sodium bicarbonate; Topical starch (10 to 98 percent); White petrolatum (greater than 30 percent); Zinc acetate (0.1 to 2 percent); Zinc carbonate (0.2 to 2 percent); and Zinc oxide (1 to 25 percent).

The compositions of the invention may further include insect repelling components. The most widely used active agent for personal care products is N,N-Diethyl-m-toluamide, frequently called “DEET” and available in the form of a concentrate containing at least about 95 percent DEET. Other synthetic chemical repellents include dimethyl phthalate, ethyl hexanediol, indalone, di-n-propylisocinchoronate, bicycloheptene, dicarboximide and tetrahydrofuraldehyde. Certain plant-derived materials also have insect repellent activity, including citronella oil and other sources of citronella (including lemon grass oil), limonene, rosemary oil and eucalyptus oil. Choice of an insect repellent for incorporation into the sunscreen emulsion will frequently be influenced by the odor of the repellent. The amount of repellent agent used will depend upon the choice of agent; DEET is useful at high concentrations, such as up to about 15 percent or more, while some of the plant-derived substances are typically used in much lower amounts, such as 0.1 percent or less.

Suitable emulsifiers or surfactants include pharmaceutically acceptable, non-toxic, non-ionic, anionic and cationic surfactants. Examples of suitable non-ionic surfactants include glycerol fatty acid esters such as glycerol monostearate, glycol fatty acid esters such as propylene glycol monostearate, polyhydric alcohol fatty acid esters such as polyethylene glycol (400) monooleate, polyoxyethylene fatty acid esters such as polyoxyethylene (40) stearate, polyoxyethylene fatty alcohol ethers such as polyoxyethylene (20) stearyl ether, polyoxyethylene sorbitan fatty acid esters such as polyoxyethylene sorbitan monostearate, sorbitan esters such as sorbitan monostearate, alkyl glycosides such as cetearyl glucoside, fatty acid ethanolamides and their derivatives such as the diethanolamide of stearic acid, and the like. Examples of suitable anionic surfactants are soaps including alkali soaps, such as sodium, potassium and ammonium salts of aliphatic carboxylic acids, usually fatty acids, such as sodium stearate. Organic amine soaps include organic amine salts of aliphatic carboxylic acids, usually fatty acids, such as triethanolamine stearate. Metallic soaps include salts of polyvalent metals and aliphatic carboxylic acids, usually fatty acids, such as aluminium stearate. Other classes of suitable anionic surfactants include sulfated fatty acid alcohols such as sodium lauryl sulfate, sulfated oils such as the sulfuric ester of ricinoleic acid disodium salt, and sulfonated compounds such as alkyl sultonates including sodium cetane sulfonate, amide sulfonates such as sodium N-methyl-N-oleyl laurate, sulfonated dibasic acid esters such as sodium dioctyl sulfosuccinate, alkyl aryl sulfonates such as sodium dodecylbenzene sulfonate, alkyl naphthalene sulfonates such a sodium isopropyl naphthalene sulfonate, petroleum sultonate such as aryl napthalene with alkyl substitutes. Examples of suitable cationic surfactants include amine salts such as octadecyl ammonium chloride, quartemary ammonium compounds such as benzalkonium chloride.

An emollient is an oleaginous or oily substance which helps to smooth and soften the skin, and may also reduce its roughness, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. Preferably, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂-C₁₅ Alkyl Benzoate sold under the trade name Finsolv TN from Finetex. One or more emollients may be present ranging in amounts from about 1 percent to about 10 percent by weight, preferably about 5 percent by weight. Another suitable emollient is DC 200 Fluid 350, a silicone fluid, available Dow Corning Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linotenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

A humectant is a moistening agent that promotes retention of water due to its hygroscopic properties Suitable humectants include glycerin, polymeric glycols such as polyethylene glycol and polypropylene glycol, mannitol and sorbitol. Preferably, the humectant is Sorbitol, 70% USP or polyethylene glycol 400, NF. One or more humectants can optionally be included in the formulation in amounts from about 1 percent to about 10 percent by weight, preferably about 5 percent by weight.

A dry-feel modifier is an agent which when added to an emulsion, imparts a “dry feel” to the skin when the emulsion dries. Dry feel modifiers can include talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A. Another dry feel modifier is an epichlorohydrin cross-linked glyceryl starch of the type that is disclosed in U.S. Pat. No. 6,488,916.

It may be advantageous to incorporate additional thickening agents, such as, for instance, various Carbopols available from Noveon Co. Particularly preferred are those agents which would not disrupt the lamellar structure in the formulation of the final product, such as non-ionic thickening agents. The selection of additional thickening agents is well within the skill of one in the art.

An “antioxidant” is a natural or synthetic substance added to the sunscreen to protect from or delay its deterioration due to the action of oxygen in the air (oxidation). They may also reduce oxidation reactions in skin tissue. Anti-oxidants prevent oxidative deterioration which may lead to the generation of rancidity and nonenyzymatic browning reaction products. Typical suitable antioxidants include propyl, octyl and dodecyl esters of gallic acid, butylated hydroxyanisole (BHA, usually purchased as a mixture of ortho and meta isomers), butylated hydroxytoluene (BHT), green tea extract, uric acid, cysteine, pyruvate, nordihydroguaiaretic acid, Vitamin A, Vitamin E and Vitamin C and their derivatives. One or more antioxidants can optionally be included in the sunscreen composition in an amount ranging from about 0.001 to about 5 weight percent, preferably about 0.01 to about 0.5 percent.

“Chelating agents” are substances used to chelate or bind metallic ions, such as with a heterocylic ring structure so that the ion is held by chemical bonds from each of the participating rings. Suitable chelating agents include ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, or combinations of any of these.

“Fragrances” are aromatic substances which can impart an aesthetically pleasing aroma to the sunscreen composition. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modern trend is to use synthetically prepared fragrances, particularly in high-volume products. One or more fragrances can optionally be included in the sunscreen composition in an amount ranging from about 0.001 to about 5 weight percent, preferably about 0.01 to about 0.5 percent by weight, Additional preservatives may also be used if desired and include well known preservative compositions such as benzyl alcohol, phenyl ethyl alcohol and benzoic acid, diazolydinyl, urea, chlorphenesin, iodopropynyl and butyl carbamate, among others.

In certain embodiments, the invention also provides non-foaming aerosol emulsion formulations. In this aspect of the invention, the emulsion formulations are maintained at a certain viscosity and/or are particularly formulated such that the propellant and the external phase of the emulsion are compatible. Viscosity of the formulations can be optimized by formulating with ingredients that will form low viscosity emulsions. In the practice of the invention, the viscosity of the formula should be sufficient to maintain a uniform emulsion without separation while being made. It should also be thin enough to be dispensed into the container. Further, the viscosity of the propellant plus formula must be thin enough to spray as a fine mist. In additional aspects of the invention, non-polar propellants are preferred for formulations with hydrocarbon external phase, such as with water in oil emulsions. Examples of such non-polar propellants include hydrocarbon and halogenated hydrocarbon, such as chlorinated and fluorinated hydrocarbon, propellants. For aqueous external phase formulations, such as for oil-in-water emulsions, more polar propellants are preferred, such as dimethyl ether and methyl ethyl ether.

In one aspect of the practice of the invention, the non-foaming aerosol emulsion formulation is prepared such that the viscosity of the emulsion at less than about 10,000 cps. In certain preferred embodiments, the viscosity of the emulsion is maintained less than about 5,000 cps, less than about 3,000 cps, or less than about 2,000 cps. In additional embodiments the viscosity is between about 3000 and about 4000 cps.

The non-foaming formulations are preferably dispensed from containers containing vapor tap valve systems to allow for additional blending of propellant and formulation upon actuation to create a light mist. Vapor taps valve systems are well known in the art and certain aspects are discussed in U.S. Pat. Nos. 4,117,958, 6,824,079, and 7,014,127, the contents of which are incorporated herein by reference. Non-vapor tap valve systems can also be used provided that the valve system contains a combination of valve and actuator orifice that produces a mist. In particular, the valve system should allow for turbulent flow to prevent clogging of the valve.

The invention will be further described by means of the following example, which is not intended to limit the invention, as defined by the appended claims, in any manner.

### EXAMPLES

A sunscreen lotion formulation containing methylparaben in an aqueous formulation at pH 8 was prepared and converted into an aerosol containing 30% w/w dimethyl ether. As described below, methylparaben in the aerosol formulation was shown to be unexpectedly stable for an extended period of time at elevated temperatures even though the pH was above the level where methylparaben should degrade.

The formula ingredients were as follows:

| | Percent Wt. | Percent Wt. |
|---|---|---|
| | Aqueous | Aerosol |
| Ingredient | Composition | Composition |
| Vitamin E, DL Alpha tocopherol | 0.05000 | 0.03500 |
| Benzyl alcohol | 0.50000 | 0.35000 |
| Fragrance | 0.25000 | 0.17500 |
| Avobenzone | 3.00000 | 2.10000 |
| Dow Corning 200 Fluid 350 CST | 0.40000 | 0.28000 |
| Disodium EDTA | 0.01000 | 0.00700 |
| Octisalate, USP | 5.00000 | 3.50000 |
| Octocrylene, USP | 2.00000 | 1.40000 |
| PVP/Eicosene copolymer | 2.00000 | 1.40000 |
| Homosalate, USP | 13.00000 | 9.10000 |
| Crill 6 | 0.71000 | 0.49700 |
| Cremophor GS-32 | 0.29000 | 0.20300 |
| Pemulen TR-2 | 0.10000 | 0.07000 |
| Methylparaben, NF | 0.20000 | 0.14000 |
| Triethanolamine, 99% NF | 0.90000 | 0.63000 |
| Propylparaben, NF | 0.10000 | 0.07000 |
| Styrene/Acrylates copolymer | 3.00000 | 2.10000 |
| Sorbitol Solution, 70% USP | 5.00000 | 3.50000 |
| Stearic Acid, NF Trip Pressed | 1.20000 | 0.84000 |
| Oxybenzone, USP | 4.00000 | 2.80000 |
| Dimethyl ether (CM) | — | 30.0000 |
| USP Purified Water | 58.29000 | 40.80300 |
| Total Weight Percent | 100 | 100 |

The aqueous emulsion formulation was prepared by combining all of the organic ingredients, including all of the sunscreens, the two parabens and the emulsifiers and heating the mixture to 160-180° F. until all of the solid components dissolve. Then water at 150-170° F. was added with stirring. The sorbitol and EDTA were added and then the formula was neutralized by adding the triethanolamine and then stirred to form a stabilized solution. The formula was allowed to cool to 110-115° F. and then the benzyl alcohol and fragrance were added. To prepare the aerosol formulation, the emulsion was allowed to cool to room temperature and then weighed into aluminum aerosol cans for the stability study or into glass bottles for the pH investigation described below. The appropriate valve, dip tube and actuator are placed in the can or bottle and the system is crimped and sealed using a hand crimper designed for the specific diameter of the can or bottle. The sealed assembly was then weighed and immersed in an ice bath. While the assembly was maintained at 0° C., the propellant was introduced into the can or bottle from a gas cylinder containing the dimethyl ether by depressing the actuator stem and allowing the gas to flow into the aerosol can or bottle. This transfer occurs because the pressure inside the aerosol can or bottle at 0° C. is less than the pressure in the gas cylinder at ambient temperature. The can or bottle was periodically weighed to determine the amount of propellant that had been added. In the final formulation shown in the example above, 70% by weight is the sunscreen emulsion and 30% by weight is dimethyl ether. The formulation also demonstrated unique characteristics for an emulsion under pressure in that the formulation produced a light mist upon expiration from a container under pressure, due to its formulation at low viscosity.

### Stability Study

Tables 1 and 2 show the results of a four week accelerated informal stability study at 50° C. and an eight week accelerated informal stability study at 40° C. Both the aerosol and the aqueous emulsion were placed in controlled temperature chambers for the duration of these stability studies. The analytical method for the assay of methylparaben uses high performance liquid chromatography (HPLC) with a reverse-phase column and a gradient mobile phase of methanol/water/acetic acid to achieve separation. The column is a Zorbax Bonus-RP, which is a C₁₄ packing with an embedded amide group to give the column unique selectivity. Detection is by UV absorbance at a wavelength of 254 nm. The amount of methylparaben determined at each time/temperature point was compared to the initial amount of methylparaben that was in the sample at time zero. As can be seen in Tables 1 & 2, the amount of methylparaben present in the aerosol formulation does not decrease under these condition whereas the methylparaben does decrease in the aqueous emulsion.

Reaction rates are usually accelerated at elevated temperatures. Stability studies at 40° C. and 50° C. are traditionally performed in order to predict and forecast room temperature stability. For example, samples which are stable at 40° C. for three months are traditionally given a room temperature expiration date of 2 years. Although this particular stability study was not carried out for 3 months at 40° C., the fact that the 2 month data at 40° C. shows no significant decrease in the amount of methylparaben present indicates that the reaction rate for the hydrolysis of methylparaben in the aerosol formulation is very small at 40° C. Those of ordinary skill in the art would recognize that these tests support the conclusion that methylparaben in this aerosol formulation will be stable at room temperature for at least 2 years. This stability of methylparaben in the aerosol formulation over an eight week period as compared to the instability of methylparaben in the aqueous emulsion is indeed a surprising and unexpected result.

TABLE 1
Stability of Methylparaben (MPB) in Solution at 50° C.

| | Initial | 2 wk @ | 4 wk @ |
|---|---|---|---|
| Formula | MPB | 50 C. | 50 C. |
| Aerosol | 100 | 97.7 | 101.8 |
| Formulation | | | |
| Aqueous | 100 | 89.1 | 79.7 |
| Emulsion | | | |

TABLE 2
Stability of Methylparaben (MPB) in Solution at 40° C.

| | Initial | 4 wk @ | 8 wk @ |
|---|---|---|---|
| Formula | MPB | 40° C. | 40° C. |
| Aerosol | 100 | 100.6 | 99.1 |
| Formulation | | | |
| Aqueous | 100 | 85.0 | NA |
| Emulsion | | | |

### pH Measurements

Since methylparaben degradation is pH dependent, a study was undertaken to see if as an aerosol, the formula ph changes during the course of the stability study. The emulsion formulation containing a few drops of Bromthymol blue indicator was placed in an aerosol glass bottle and sealed. Dimethyl ether was then added as described earlier. Bromthymol blue indicator is yellow in acid, green at pH 8 and blue in base. The initial color of the indicator in the aerosol formulation was green and after six weeks at 50° C. exposed to the contents of the aerosol bottle the indicator was stilt green in color. This confirms that the pH of the aerosol remained close to pH 8 during the six week test period

Although certain presently preferred embodiments of the invention have been described herein, it will be apparent to those skilled in the art to which the invention pertains that variations and modifications of the described embodiments may be made without departing from the spirit and scope of the invention.

What is claimed is:

1. An aerosol composition comprising methylparaben as a preservative and having a pH at or above a level where methylparaben would normally hydrolyze, wherein the methylparaben does not significantly degrade.

2. The aerosol composition of claim 1, wherein the composition comprises an aerosol propellant chosen from the group consisting of C₁ to C₅ hydrocarbons hydrofluorocarbons (HFCs), chlorofluorocarbons (CFCs), nitrogen, ethers and mixtures thereof.

3. The aerosol of claim 2 wherein the C₁ to C₅ hydrocarbon is chosen from methane, ethane, propane, isopropane, butane, isobutane, butene, pentane, isopentane, neopentane, pentene, and mixtures thereof.

4. The aerosol of claim 2, wherein the propellant is dimethyl ether.

5. The aerosol composition of claim 1 in the form of a foam or mousse.

6. The aerosol composition of claim 1 further comprising one or more cosmetic ingredients chosen from the group consisting of absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents, skin protectants, solvents, foam boosters, hydrotropes, solubilizing agents, suspending agents, sunscreen agents, ultraviolet light absorbers, SPF boosters, waterproofing agents, aqueous and nonaqueous viscosity increasing agents.

7. An aqueous composition under pressure comprising a propellant in admixture and methylparaben, wherein the composition maintains a pH of greater than 6 and the methylparaben does not significantly degrade.

8. The composition of claim 7 wherein the composition maintains a pH of greater than 7.

9. The composition of claim 7, wherein the composition maintains a pH of greater than 8.

10. The composition of claim 7, wherein the methylparaben does not significantly degrade for a period of at least two years.

11. The aerosol composition of claim 7, wherein the composition comprises an aerosol propellant chosen from the group consisting of C₁ to C₅ hydrocarbons hydrofluorocarbons (HFCs), chlorofluorocarbons (CFCs), nitrogen, ethers and mixtures thereof.

12. The aerosol of claim 11 wherein the C₁ to C₅ hydrocarbon is chosen from methane, ethane, propane, isopropane, butane, isobutane, butene, pentane, isopentane, neopentane, pentene, and mixtures thereof.

13. The aerosol of claim 11, wherein the propellant is dimethyl ether.

14. A sunscreen composition comprising methylparaben in an amount sufficient to act as an antimicrobial agent, wherein the composition comprises a pH above 6 and the methylparaben does not significantly degrade, and wherein the composition is contained in a pressurized container.

15. A method for preserving an aqueous composition under pressure and having a pH over 6, comprising adding to the composition of preservative system comprising methylparaben in an antimicrobial effective amount.

16. The method of claim 15, wherein the methylparaben is added to the composition before it is pressurized.

17. The method of claim 15, wherein the composition is pressurized by adding a propellant.

18. The method of claim 17, wherein the propellant comprises an aerosol propellant chosen from the group consisting of C₁ to C₅ hydrocarbons hydrofluorocarbons (HFCs), chlorofluorocarbons (CFCs), nitrogen, ethers and mixtures thereof.

19. The method of claim 18 wherein the C₁ to C₅ hydrocarbon is chosen from methane, ethane, propane, isopropane, butane, isobutane, butene, pentane, isopentane, neopentane, pentene, and mixtures thereof.

20. The method of claim 18, wherein the propellant is dimethyl ether.

21. A preservative system for an aqueous aerosol composition having a pH at or above a level where methylparaben would begin to hydrolyze, the preservative system comprising methylparaben present in an antimicrobial effective amount.

22. The preservative system of claim 21, wherein the composition has a pH of greater than 7.

23. The preservative system of claim 21, wherein the composition has a pH of greater than 8.

24. The preservative system of claim 21, wherein the methylparaben does not significantly degrade for a period of at least two years.

25. The preservative system of claim 21, wherein the system is contained in an aerosol composition chosen from the group consisting of moisturizers, cleansers, conditioners, shampoo, body wash, styling gel/lotion, eye cream and eye liner, blush, mascara, foundation, nail polish, polish remover, eye shadow, lipstick, lip gloss, lip liners, lip balms, makeup remover, nail treatment, foot care compositions, acne treatment, redness/rosacea treatment, varicose/spider vein treatment, anti-aging compositions, sunscreens, sunless tanning compositions, after-sun compositions, concealers, hair color and bleaching compositions, skin fading/lighteners, body firming lotion, shaving cream, after shave, relaxer, antiperspirants and deodorants, exfoliants, scrubs, liquid hand soap, bubble bath, pain and wound treatment compositions, insect repellant, anti-itch and rash cream, styling mousse and foams, bath oils and salts, toothpaste, perfume, glitter, lubricants, body powder, body oil, body spray, baby lotion, diaper cream, baby soap, baby shampoo, baby oil, baby wipes, hair-loss treatment, hair spray, cuticle treatment, dandruff/scalp treatment, depilatory, hair growth inhibitors, hair removal waxes, personal cleansing, cologne, oil controller, hand sanitizer, mouthwash, tooth whitening, eye drops, and artificial tears compositions.

26. A sunscreen composition comprising methylparaben in an amount sufficient to act as an antimicrobial agent, wherein the composition comprises a pH above 6 and the methylparaben does not significantly degrade and wherein the composition is contained in a container pressurized with dimethyl ether.

27. An aerosol emulsion formulated to be held in a container under pressure, wherein the emulsion formulation does not form a foam upon expression from the container.

28. The emulsion formulation of claim 27, wherein the emulsion is an oil-in-water emulsion.

29. The emulsion formulation of claim 27, wherein the emulsion is a water-in-oil emulsion.

30. The emulsion formulation of claim 28, wherein the formulation comprises a non-polar propellant.

31. The emulsion formulation of claim 30, wherein the non-polar propellant is chosen from the group consisting of hydrocarbon and halogenated hydrocarbon propellants.

32. The emulsion formulation of claim 29, wherein the formulation comprises a polar propellant.

33. The emulsion formulation of claim 32, wherein the polar propellant is chosen from the group consisting of dimethyl ether and methyl ethyl ether.

34. The emulsion formulation of claim 27, wherein in the formulation is prepared at a viscosity of less than about 10,000 cps.

35. The emulsion formulation of claim 27, wherein in the formulation is prepared at a viscosity of less than about 5,000 cps.

36. The emulsion formulation of claim 27, wherein in the formulation is prepared at a viscosity of less than about 3,000 cps.

37. The emulsion formulation of claim 27, wherein in the formulation is prepared at a viscosity of less than about 2,000 cps.

38. The emulsion formulation of claim 27, wherein in the formulation is prepared at a viscosity of between about 3,000 cps and 4,000 cps.

39. A container comprising the emulsion formulation of claim 27 maintained under pressure.

40. The container of claim 39 further comprising a vapor tap valve system.

41. The container of claim 40 further comprising a valve system allowing for turbulent flow of the emulsion upon expression from the container.

42. The emulsion formulation of claim 27, wherein the formulation comprises a sunscreen composition.
